(19) United States
(12) Patent Application Publication
(10) Pub. No.: US 2023/0374432 A1
(43) Pub. Date: Nov. 23, 2023

(54) BIOREACTOR SYSTEMS AND METHODS FOR CULTURING CELLS

(71) Applicant: TurtleTree Labs Pte. Ltd., Singapore (SG)

(72) Inventors: Henry GOH, Singapore (SG); Ying Ying WU, Singapore (SG); Gen YONG, Singapore (SG)

(21) Appl. No.: 18/200,121

(22) Filed: May 22, 2023

Related U.S. Application Data
(63) Continuation of application No. PCT/SG2021/050704, filed on Nov. 16, 2021.
(60) Provisional application No. 63/117,557, filed on Nov. 24, 2020.

(51) Int. Cl. C12M 1/12; C12M 1/04

(52) U.S. Cl. CPC C12M 25/14; C12M 23/24; C12M 25/10

(57) ABSTRACT

In particular, the present disclosure relates to apparatus, systems and methods for culturing cells, e.g., mammalian cells, and in particular instances, mammary cells. The present disclosure describes the use of various 3D cell scaffolds, in particular hollow fiber bioreactors, for the cell seeding, proliferation and differentiation of the cells (e.g., mammalian cells such as mammary cells). The use of magnetic mechanisms for moving the cell scaffold relative to the bioreactor vessel is disclosed.

### CROSS REFERENCE

This application is a continuation of International Application No. PCT/SG2021/050704, filed Nov. 16, 2021, which claims the benefit of U.S. Provisional Patent Application No. 63/117,557, filed Nov. 24, 2020, each of which is incorporated by reference herein in its entirety for all purposes.

### BACKGROUND

The present disclosure relates to apparatus, systems and methods for culturing cells. In particular, the present disclosure relates to apparatus, systems and methods for culturing mammalian cells, and particularly, mammary cells, including hollow fiber bioreactors.

### SUMMARY

The summary is provided to introduce aspects of some embodiments of the present disclosure and claimed invention in a simplified form, and is not intended to identify key or essential elements of the claimed invention, nor is it intended to limit the scope of the claims.

It is to be understood that the present invention may include a variety of different embodiments, and this Summary is not meant to be limiting or all-inclusive. This Summary provides some general descriptions of features that may be included in embodiments, and also include some more specific descriptions of other features that may be included in other embodiments.

In some aspects, the present disclosure relates to apparatus, systems and methods for culturing cells, e.g., mammalian cells, and in particular instances, mammary cells. In an embodiment, an apparatus is disclosed, wherein the apparatus comprises a vessel having an exterior surface and an interior surface defining a sealed cell culture chamber, the vessel having a longitudinal axis, and a plurality of fluid inlet ports and a plurality of fluid outlet ports; a cell scaffold secured to a mount, said mount and said cell scaffold disposed within the sealed cell culture chamber and configured to move along the longitudinal axis relative to the vessel, said mount including a magnetically responsive material; and one or more external magnets disposed along the exterior surface of the vessel and configured to magnetically engage the mount. In an embodiment, the apparatus includes the mount and the cell scaffold that are configured to be selectively movable between at least a first position and a second position relative to the vessel along the longitudinal axis. In a further embodiment, in the apparatus, the vessel can be cylindrical in shape and can have a top end and a bottom end, wherein the vessel bottom end is removably fixed to a base mechanism, and the top end is removable. In some embodiments, the base mechanism is configured to move relative to the mount and cell scaffold. The apparatus can include an embodiment where the mount is configured to be selectively movable relative to the vessel between at least a first position and a second position along the longitudinal axis, and wherein movement of the one or more external magnets between the first position and the second position is capable of moving the mount within the cell culture chamber between the first position and the second position.

Another embodiment described herein relates to a cell scaffold cartridge. The cell scaffold cartridge can comprise a mount configured to be disposed within a cell culture chamber of a vessel, and configured to move along the longitudinal axis of the vessel relative to the vessel, the mount including a magnetically responsive material; and a cell scaffold secured to said mount. In the cell scaffold cartridge, the mount can have a top surface and a bottom surface, wherein the cell scaffold is removably secured to the mount bottom surface by magnetic mechanisms. The cell scaffold can be secured to the mount bottom by any removably secured mechanisms, such as nuts, bolts, clamps, screws, etc. In an embodiment, the cell scaffold cartridge can include one or more of a plurality of hollow fibers, flat sheet membrane, matrix, cage with macro carriers, porous membrane bag with macro carriers, or any combination thereof.

An embodiment described in the present disclosure includes a cell culture system including two or more bioreactors. A bioreactor of the two or more bioreactors may comprise a vessel having an exterior surface and an interior surface defining a sealed cell culture chamber, the vessel having a longitudinal axis, and a plurality of fluid inlet ports and a plurality of fluid outlet ports; a cell scaffold secured to a mount, said mount and said cell scaffold disposed within the sealed cell culture chamber and configured to move along the longitudinal axis relative to the vessel, said mount including a magnetically responsive material; and one or more external magnets disposed along the exterior surface of the vessel and configured to magnetically engage the mount upon coupling to the magnetically responsive material. The cell culture system described herein can include a system wherein the mount and the cell scaffold are configured to be selectively movable between at least a first position and a second position relative to the vessel along the longitudinal axis. In an embodiment, the cell culture system described herein can include a vessel that is cylindrical in shape and has a top end and a bottom end, wherein the vessel bottom end is removably fixed to a base mechanism, and the top end is removable. The present disclosure includes a cell culture system wherein the base mechanism is configured to move relative to the mount and the cell scaffold. In a further embodiment, the mount is configured to be selectively movable relative to the vessel between at least a first position and a second position along the longitudinal axis, and wherein movement of the one or more external magnets between the first position and the second position is capable of moving the mount within the cell culture chamber between the first position and the second position.

Described herein are methods, and in particular, a method of culturing cells in one or more bioreactors. In an embodiment, wherein a bioreactor can include a vessel having an exterior surface and an interior surface defining a sealed cell culture chamber including a liquid medium, the vessel having a longitudinal axis, and a plurality of fluid inlet ports in fluid communication with the cell culture chamber and a plurality of fluid outlet ports in fluid communication with the cell culture chamber; and a cartridge including a cell scaffold secured to a mount, said cell scaffold including the cells adhered thereto, said mount disposed within the sealed cell culture chamber and configured to move along the longitudinal axis relative to the vessel and configured to at least partially dispose the cell scaffold in the liquid medium, the mount secured to a first external mechanism, and the vessel secured to a second external mechanism, the method comprises controllably moving at least one of the first external mechanism and the second external mechanism relative to each other such that the mount and cell scaffold move along the longitudinal axis of the vessel. In a method described herein, fresh culture media can be provided via at least one of the plurality of fluid inlet ports, and substantially spent culture media is removed via at least one of the plurality of fluid outlet ports. In various embodiments, the cell scaffold can include one or more of a plurality of hollow fibers, flat sheet membrane, encapsulated, matrix, cage with macro carriers, porous membrane bag with macro carriers, or any combination thereof. In an embodiment of a method described herein, at least one of the first external mechanisms and at least one of the second external mechanisms can be moved relative to each other such that the mount and cell scaffold move along the longitudinal axis of the vessel. The described methods include moving the second external mechanism while the first external mechanism remains stationary. It is contemplated that the cells can be seeded, proliferated or differentiated in one or more isolated bioreactors, or within fluidically connected bioreactors. The fluidically connected bioreactors may share a same, or substantially similar (e.g., homogeneous) environment (e.g., similar nutrient compositions and/or concentrations, waste concentration, gas concentration, etc.). For example, the connected bioreactors may share a gas headspace, via connection or a gas supplied through inlet ports of the connected bioreactors. In some instances, the fluidically connected bioreactors may be fluidically isolated during or prior to performing a cell process (e.g., cell seeding, sample extraction, etc.).

In an embodiment, the mount includes a top end and a bottom end, the bottom end of the mount can be removably secured to the cell scaffold. Further, the mount can include a magnetically responsive material, and the first external mechanism includes one or more magnets positioned external to the vessel that magnetically engage the mount. In some embodiments, the top end of the vessel includes a magnet and is configured to magnetically engage the mount with a stronger force than the first external magnets.

In another aspect, disclosed herein is a method, comprising (a) providing: (i) a vessel comprising a first fluid and a second fluid, wherein the first fluid is different from the second fluid and (ii) a cell scaffold comprising at least one cell; (b) moving the cell scaffold to a first position within the vessel to expose the at least one cell to the first fluid; and (c) moving the cell scaffold to a second position within the vessel to expose the at least one cell to the second fluid, wherein the first position and the second position are different.

In some embodiments, the first fluid is a liquid. In some embodiments, the liquid is cell culture media. In some embodiments, the second fluid is a gas. In some embodiments, in the second position, the cell scaffold is at least partially removed from the cell culture media. In some embodiments, the vessel comprises a longitudinal axis, and the first position and the second position are different positions along the longitudinal axis. In some embodiments, the longitudinal axis is a vertical axis. In some embodiments, the cell scaffold is coupled to a magnet, wherein the magnet is configured to move the cell scaffold from the first position to the second position. In some embodiments, the magnet is configured to move the cell scaffold from the second position to the first position. In some embodiments, the vessel comprises a longitudinal axis, and the first position and the second position are different positions along the longitudinal axis. In some embodiments, the cell scaffold is coupled to a mount comprising the magnet. In some embodiments, the mount is removably coupled to the cell scaffold. In some embodiments, (a) further comprises providing one or more external magnets disposed along an exterior surface of the vessel, wherein the one or more external magnets is configured to magnetically engage the mount. In some embodiments, (b) and (c) are performed using the one or more external magnets. In some embodiments, the vessel comprises a fluid inlet port and a fluid outlet port. In some embodiments, the vessel is coupled to a movement actuator. The movement actuator can be, for example, a hydraulic actuator, a pneumatic actuator, an electric actuator, a thermal actuator, a mechanical actuator and a magnetic actuator. In some embodiments, the vessel comprises or is coupled to a movement actuator selected from the group consisting of: a hydraulic actuator, a pneumatic actuator, an electric actuator, a thermal actuator, a mechanical actuator and a magnetic actuator. In some embodiments, (b) and (c) are performed using the movement actuator.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are incorporated by reference herein to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 is an illustration showing an embodiment of a bioreactor as described herein.

FIG. 2 illustrates an embodiment of a bioreactor as described herein, with the movement of the cell scaffold relative to the bioreactor vessel.

FIG. 3 illustrates an exploded view of the reactor chamber.

FIG. 4 illustrates various substrate or cell scaffold cartridges.

FIG. 5 illustrates two alternatives for a cell scaffold cartridge as described herein.

FIG. 6 illustrates an exploded view of an embodiment for an assembly for a bioreactor, excluding the reaction chamber.

FIG. 7 illustrates an assembly of a cell scaffold mount and external holder components.

FIG. 8 is an illustration of an assembly of a cell scaffold mount and external holder components.

FIG. 9 illustrates a sectioned view of the assembly of FIG. 8.

FIG. 10 is an illustration of four bioreactor assemblies connected to a drive mechanism.

FIG. 11 provides an example workflow as described herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

It should be understood that the specific order or hierarchy of steps in the processes or methods disclosed herein is an example. Based upon design preferences, it is understood that the specific order or hierarchy of steps in the processes or methods may be rearranged while remaining within the scope of the present disclosure. Any accompanying method claims present elements of the various steps in a sample order and are not meant to be limited to the specific order or hierarchy presented.

Disclosed herein is an apparatus that includes a vessel having an exterior surface and an interior surface defining a sealed cell culture chamber, the vessel having a longitudinal axis, and a plurality of fluid inlet ports and a plurality of fluid outlet ports; a cell scaffold secured to a mount, said mount and said cell scaffold disposed within the sealed cell culture chamber and configured to move along the longitudinal axis relative to the vessel, said mount including a magnetically responsive material; and one or more external magnets disposed along the exterior surface of the vessel and configured to magnetically engage the mount upon coupling to the magnetically responsive material.

In an embodiment, the cell scaffold can be any type of structure whereby cells can adhere to a surface and be cultured to grow, proliferate or differentiate. 3D cell scaffolds, which can be made of polymeric biomaterials, have the advantage of providing a structural support for cell attachment and tissue development. Cell scaffolds can allow for recapitulation of the extracellular environment of cells by providing attachment sites, the ability for cells to grow in 3D shape, and for some of them, rigidity or other biophysical cues of this environment and associated soluble factors (e.g., growth factors, paracrine signaling, immune system signals, etc.). This is in contrast to traditional 2D cell cultures in which cells are grown in a flat monolayer on a plate. The present invention contemplates the use of either 2D or 3D scaffolds and their respective use will depend on the user. 3D cell cultures can be grown with or without a supporting scaffold. 3D cell culture may be performed within a supporting scaffold to allow growth in all directions. Types of scaffold may include hydrogels: Polymeric material containing a network of crosslinked polymer chains that can absorb and retain water. Hydrogels can be derived from animals (e.g., Matrigel®, collagen, gelatin, hyaluronic acid, or other polymeric material or peptide) or plants or algae, (e.g., alginate, agarose), or synthesized from chemicals (e.g., QGel® Matrix, acrylamide and bis-acrylamide, polymethyl methacrylate). A hydrogel may comprise one or more polymer chains and may comprise one or more polymer groups (e.g., copolymer, block polymer). A hydrogel may be functionalized, e.g., using crosslinkers, such as heterobifunctional crosslinkers (e.g., NETS-ester, Sulfo-SANPAH, etc.) to link a protein or peptide (e.g., collagen, Matrigel®, hyaluronic acid, RGD peptide, etc.); Inert matrices: Sponge-like membranes made of polymers (e.g., polystyrene) which contain pores for cells to proliferate and grow. In some embodiments, the bioreactor may comprise a hollow fiber system, which can deliver media to the cells in a manner akin to the delivery of blood through the capillary networks in vivo.

The hollow fiber bioreactor design may be used in the production of several different products, e.g., secreted products, proteins, sugars, lipids, etc., from cells, e.g., mammalian cells, for the production of cells and in tissue engineering applications, such as bioartificial organs. The possibility of maintaining these systems at near tissue densities can result in an increased per cell productivity, making high concentrations of both products and cells available. Cells may be inoculated or seeded outside the fibers in the extracapillary (EC) space; medium is circulated from a reservoir, through the fiber intracapillary (IC) space, and returned to the reservoir afterwards. The semi-permeable fiber membrane may be characterized as ultrafiltrative (molecular weight cutoff of 10-100 kDa) or microporous (0.1-0.2 μm pores). In an embodiment, the plurality of hollow fibers are semipermeable and define an intracapillary space and an extracapillary space, wherein the intracapillary space is configured to contain a first fluid, and the extracapillary space is configured to contain a second fluid.

Hollow fiber bioreactors may resemble the capillary network in vivo and deliver nutrients and other required molecules in a fast, efficient and reliable fashion. Their high surface area to occupied volume ratio allows the delivery of these molecules, and that is particularly true in cases where the overall volume does matter, such as in attempts for scaling-up. In addition to the use of hollow fibers, other systems can be used, for example, a flat sheet membrane, matrix, cage with macro carriers, or a porous membrane bag with macro or micro carriers, any of which may include a surface configured to receive cell seeding.

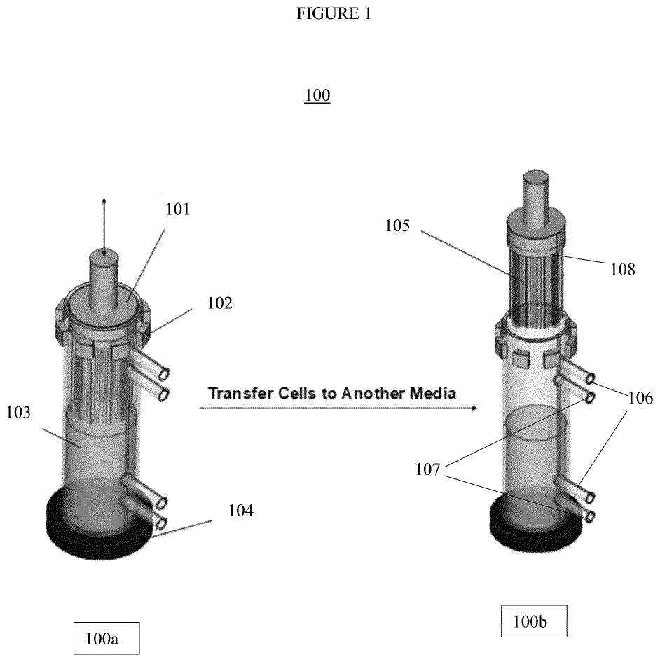

In FIG. 1, an illustration is provided showing a vessel, such as a bioreactor 100 as described herein. The bioreactor 100 can include a plurality of fluid inlet ports 106 and a plurality of fluid outlet ports 107, which may be independently operable or controlled. In FIG. 1, bioreactor 100 may comprise or be coupled to a first external mechanism (not shown) and may be secured on a bottom end to a second external mechanism 104. View 100a illustrates the cell scaffold 105, which may comprise one or more cells, removed completely from a first medium, such as liquid cell culture media 103. Cell scaffold 105 can be removably secured to mount 108. Mount 108 can be secured to a top end 101 of the vessel by any mechanism, such as removable fixtures, such as clamps, screws, form-fitting pairs, hooks and loops, latches, threads, screws, staples, clips, clamps, prongs, rings, brads, rubber bands, rivets, grommets, pins, ties, snaps, velcro, adhesives (e.g., glue), tapes, vacuum, seals, a combination thereof, or any other types of fastening mechanisms. When secured to each other, top end 101, mount 108, and cell scaffold 105 can move along the longitudinal axis (e.g., vertical or substantially vertical axis) of the vessel, as indicated by the double arrow in view 100a. Top end 101 can be configured to be removably secured to the first external mechanism (not shown) that may be independent from the second external mechanism 104. The first external mechanism can be a fixed mechanism without movement, or it can be a mechanical member that can move along the same longitudinal axis (e.g., vertical or substantially vertical axis) of the vessel. Mount 108 can be secured to top end 101 by magnetic force, for example the magnetic force can be provided by an electromagnet. Cell scaffold 105 can be secured to mount 108 by removable fixtures, such as clamps, screws, and the like. Mount 108, in some embodiments, contains a magnetically responsive material such as a ferromagnet, or it can be any strong magnet, such as a neodymium magnet. The bioreactor 100 may have, proximate to the top end of the vessel, at least two strong magnets 102 that are placed around the external surface of the bioreactor. The at least two strong magnets 102 may be arranged in any useful fashion around the external surface of the bioreactor; for example, the at least two strong magnets 102 may be placed equidistant and radially along the external surface. The magnets can be fixed to a stationary support, or they can be fixed to a dynamic moveable support. In an embodiment shown in other Figures herein, the magnets can be placed in one or more support mechanisms in a ring configuration. In view 100b, the cell scaffold 105 has been removed from the liquid cell culture media 103 and from the bioreactor vessel 100 and exposed to a second medium (e.g., gas, such as air). Cells adherent to the cell scaffold 105 can be removed out of a liquid cell culture media (e.g., spent cell culture media) by removing the top end 101 (e.g., by use of the first external mechanism), thus also removing the mount 108 and cell scaffold 105. The cell culture media 103 can be replaced or refreshed; and the top end 101, mount 103 and cell scaffold 105 reinserted into the bioreactor 100. Fluid inlet ports 106 can provide an inlet for a gas or a liquid. The fluid inlet ports 106 can each have a controllable valve mechanism for controlling the flow of fluid into the bioreactor. Fluid inlet port 106 near the top end of the bioreactor can be used for inputting a second medium (e.g., gas) into the chamber. The gas can be air, an oxygen-rich gas, or an oxygen-poor gas, an inert gas, etc. The fluid inlet port 106 near the bottom end of the reactor can be for inputting cell culture media, or another liquid for cleaning, for example. Fluid outlet ports 107 can provide an outlet for a gas or a liquid. The fluid outlet ports 107 can each have a controllable valve mechanism for controlling the flow of fluid out of the bioreactor. Fluid outlet port 107 near the top end of the bioreactor can be used for releasing a gas out the chamber. The fluid outlet port 107 near the bottom end of the reactor can be for outputting spent cell culture media, or outputting a spent liquid for cleaning, for example. In some instances, the vessel comprises a sampling output port (not shown), from which a gas or liquid (e.g., cell culture media) can be aseptically extracted. In some instances, one of the fluid inlet ports or fluid outlet ports may be used as a sampling port. The fluid inlet ports 106 or fluid outlet ports 107 may be coupled to fluid handling modules or systems, e.g., aeration modules, flow regulators, liquid handling systems, etc. The vessel may be subjected to conditions for culturing cells and accordingly may be integrated with controllers for regulating temperature, humidity, oxygen content, gas pressure, fluid flow, etc.

In an embodiment, the first external mechanism that can be secured to the top end 101 can be fixed, and the second external mechanism 104 can move relative to the first external mechanism such that as the second external mechanism moves, the top end 101, mount 103 and cell scaffold 105 move in unison along the longitudinal axis of the bioreactor vessel 100. Alternatively, it is contemplated that the second external mechanism 104 is fixed, and the first external mechanism can be secured to the top end 101 and can move the top end 101, mount 103 and cell scaffold 105 in unison along the longitudinal axis of the bioreactor vessel 100. In effect, the first external mechanism and the second external mechanism move relative to one another along the longitudinal axis of the bioreactor vessel 100, thereby causing top end 101, mount 103 and cell scaffold 105 to likewise move in unison along the longitudinal axis of the bioreactor vessel 100. The longitudinal axis may be, in some instances, a vertical axis.

In an embodiment, the vessel or a component thereof or a component coupled thereto (e.g., the first external mechanism or the second external mechanism) may comprise a movement actuator. The movement actuator may be, for example, a hydraulic actuator, a pneumatic actuator, an electric actuator, a thermal actuator, a mechanical actuator, a magnetic actuator, or a combination thereof. The movement actuator may be used to move the cell scaffold along the longitudinal axis within the vessel, or along a different direction from the longitudinal axis. The movement actuator may be configured to automatically move an external mechanism.

In some instances, the movement actuator comprises magnets, which may be positioned external to the bioreactor vessel and can move relative to the bioreactor vessel itself. This can be achieved by any number of mechanisms and configurations. For example, the external magnets (e.g., as depicted in 102) can be fixably mounted to a stationary support while the vessel is fixed to a support that moves; alternatively, the external magnets can be mounted to a moveable support while the vessel is mounted to a stationary support; or in another embodiment, the external magnets can be fixed to a first movable support and the vessel fixed to a second moveable support so that both components are capable of movement. The movement of the external magnets relative to the vessel can be achieved without limitation to any particular design. In each case, the movement of the external magnets along the longitudinal axis of the vessel results in the movement of the cell scaffold, including the cells adhered thereto, along such axis. By this movement, the cells and cell scaffold can be withdrawn from the cell culture media and exposed to the gas environment of the vessel chamber for any predetermined period of time, and then reinserted into the cell culture media. The cells and cell scaffold may be configured to be disconnected and reconnected in any convenient or useful order or duration. Various control systems can be implemented to control the rate of withdrawal, the exposure time of the cells to the gas, and exposure time of the cells to the liquid cell culture media.

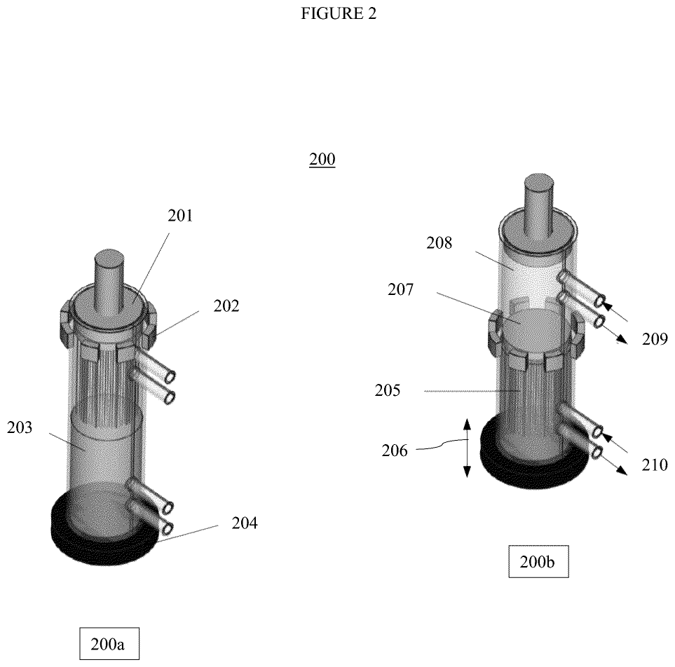

FIG. 2 illustrates an embodiment similar to FIG. 1. FIG. 2 shows a vessel, such as a bioreactor 200, as described herein. The bioreactor 200 can include a plurality of fluid inlet ports and a plurality of fluid outlet ports as shown in FIG. 1. The inlet and outlet ports are not numbered in FIG. 2 to reduce complexity. In FIG. 2, bioreactor 200 may be coupled to a first external mechanism (not shown) and may be secured on a bottom end to a second external mechanism 204. View 200a illustrates the cell scaffold 205 at least partially inserted into the liquid cell culture media 203. Cell scaffold 205 can also be viewed as being completely removed from the liquid cell culture media 203. Cell scaffold 205 is removably secured to mount 207 (e.g., at the bottom surface of the mount). Mount 207 can be secured to a top end 201 of the vessel by any mechanism or approach, as described herein. When secured to each other, mount 207, and cell scaffold 205 can move along the longitudinal axis of the vessel in unison, as indicated by the double arrow 206 in view 200b. Top end 201 can be configured to be removably secured to a first external mechanism (not shown) that is independent from the second external mechanism 204. The first external mechanism can be a fixed mechanism without movement, or it can be a mechanical member that can move along the same longitudinal axis of the vessel. Mount 207 can be secured to top end 201 by any fixing mechanism, such as magnetic force; for example, the magnetic force can be provided by an electromagnet. Cell scaffold 205 can be secured to mount 207 as described herein. Mount 207 may contain a magnetically responsive material such as a ferromagnet, or it can be any strong magnet, such as a neodymium magnet. The bioreactor 200 may have, proximate to the top end of the vessel, at least two strong magnets 202 that are placed equidistant around the external surface of the bioreactor. The magnet or magnets may be placed in a substantially circular arrangement around the exterior of the bioreactor 200. In view 200b, the cell scaffold 205 is inserted into a first media, e.g., the liquid cell culture media 203. Cells adherent to the cell scaffold 205 can expand, proliferate and differentiate, if beneficial. Cell expression products may be produced and harvested from the cell culture media 203 according to standard methods. The fluid outlet port (as shown in FIG. 1) near the bottom end of the reactor can be for outputting spent cell culture media and cell product, or outputting a spent liquid for cleaning, for example. Alternatively or in addition to, the vessel may comprise a sample port for aseptically harvesting the cell product or the cell culture media. FIG. 2 illustrates the movement of the magnets 202 which cause the mount 207 and cell scaffold 205 to move from a first position (FIG. 200a) to a second position (FIG. 200b). It is contemplated as an embodiment, that the same relative movement can be achieved by keeping the magnets 202 stationary and moving the mechanism 204 from a first position to a second position. In an embodiment, the mount 207 can include a plurality of holes, wherein each of the plurality of holes is of a size to permit the transport of a gas or a nutrient therethrough, and to prevent the transport of a cell therethrough. When the cell scaffold is withdrawn from the cell culture medium, it and the cells adherent thereto, can be exposed to a second medium, e.g., various gases, in order to assist in the growth or promotion of certain cell products. Such gases can include, air, an oxygen-rich or oxygen-poor gas, or an inert gas.

Cell culture product can be any product produced by a cell (e.g., mammalian cell) in culture. Such products can include, in non-limiting examples, proteins, peptides, antibodies, antibody fragments, hormones, polypeptides, lipids, carbohydrates, metabolites, and the like. In some embodiments, the cells are mammary cells and the cell product is a milk product. The culture of mammary cells can produce a product very similar to natural milk. The systems, apparatus and methods disclosed herein are highly suitable for large-scale and high-throughput manufacturing of such cell products.

FIG. 2 also illustrates how the second external mechanism 204 can move from one location to another, e.g., from a first position within the vessel to a second position within the vessel. For example, when the cell scaffold 205 is withdrawn from the bioreactor vessel, the vessel is fixed to the second external mechanism and the second external mechanism can move in a lateral fashion to position the bioreactor vessel in a different location. In addition, the second external mechanism 204 can include additional bioreactor vessels attached or fixed thereto which can be moved to new positions. For example, after the liquid cell culture media 203 is spent in a bioreactor vessel 200, the cell scaffold may be withdrawn from the vessel by the action of the second external mechanism, which moves the bioreactor vessel away from the cell scaffold 205 and along the longitudinal axis of the vessel until the cell scaffold 205 is no longer within the vessel. The second external mechanism 204 can move laterally, for example, to bring another bioreactor into proximity of the cell scaffold 205. When aligned, the second external mechanism can move in a longitudinal direction to bring the cell scaffold 205 into the new bioreactor vessel. The new bioreactor vessel can include fresh cell culture media or can provide a bioreactor capable of culture conditions different from the previous bioreactor vessel.

In some instances, the cell scaffold 205, and optionally the mount 202 and/or top end 201, may be configured to move within the vessel in a non-longitudinal direction. For instance, the cell scaffold 205 may be moved radially, laterally, or along a non-lateral or non-longitudinal axis. The cell scaffold 205 may rotate or otherwise be translated to a different position within or external to the vessel.

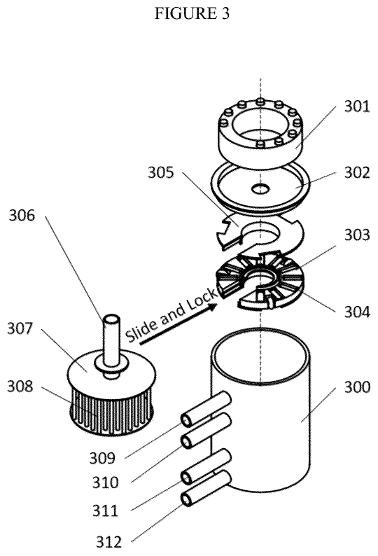

FIG. 3 illustrates an exploded view of the reactor chamber, or vessel 300, having a gas inlet port 309, a gas outlet port 310, a liquid inlet port 311 and a liquid outlet port 312. A magnetic clamp 301, reactor top cap 302, substrate mount 303, and substrate mount cover 305, which may align with the longitudinal axis of the vessel 300. Substrate mount cover 305 can be a magnetically responsive material so as to engage magnetic clamp 301. Substrate mount 303 can include an array of magnets 304 to engage the magnetic clamp 301. A cell scaffold cartridge depicted by elements 307 and 308 can be designed for a rapid replacement in the bioreactor, if useful. The cell scaffold cartridge may comprise a hollow fiber substrate 308 that is contained with a substrate cap 307 and an optional hollow tube 306 extending longitudinally from the exterior environment to the interior of the hollow fiber substrate 308. Tube 306 can be used as a mechanism for access to the hollow fiber substrate environment, for example, with a probe. Tube 306 can also be used as a mechanism for secondary flow into the substrate environment. Secondary flow can include any additional nutrients, gases, fluids and the like. Substrate mount 303 and substrate mount cover 305 can be of any design and need not be the shape and configuration depicted. In the embodiment shown in FIG. 3, substrate mount 303 and substrate mount cover 305 are “C” shaped, or substantially circular to allow for the acceptance of tube 306, as well as other substrate assemblies. The mechanism can contain an inter-locking mechanism, if useful, to lock the components securely to another. In an embodiment, the “C” shape can also allow for the flow of fluids through ports 309-312 without obstruction.

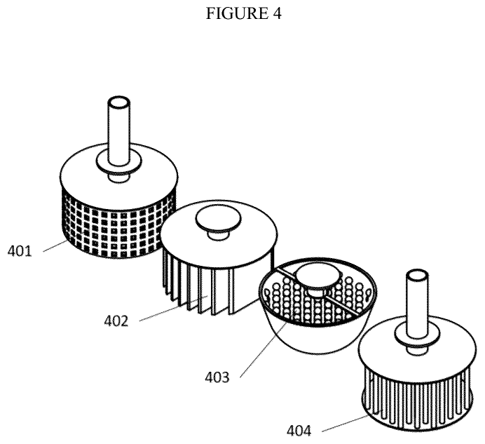

FIG. 4 illustrates various substrate or cell scaffold cartridges, which may comprise or be configured to adhere to one or more cells (e.g., via cell seeding onto the cell scaffold). A cell scaffold cartridge comprised of a cage with macro carriers is depicted as 401. A cell scaffold cartridge comprised of substrate sheets is depicted as 402. A cell scaffold cartridge comprised of a porous bag with macro carriers is depicted as 403. A cell scaffold cartridge comprised of an array, or a plurality of hollow fibers is depicted as 404. Optional tube 306 (from FIG. 3) can be included with any cartridge choice or design.

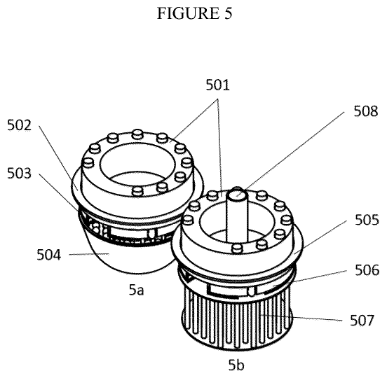

FIG. 5 illustrates two alternatives for a cell scaffold cartridge as described herein. In 5a, a cell scaffold cartridge comprising a porous bag with macro carriers is depicted. Magnetic clamp 501 can be secured to a bioreactor top end 502 through magnetic mechanisms or other techniques; and substrate mount 503 may be secured to the bioreactor top end 502. As described herein for various embodiments, a substrate mount can include magnetically responsive materials. In FIG. 5a, substrate mount 503 includes various magnets positioned around the central axis of the mount 503. Substrate mount 503 is configured to removably engage the substrate portion 504 (in this depiction, a porous bag with macro carriers). Substrate mount 503 can be configured to removably engage the substrate portion 504 through magnetic mechanisms. In 5b, a cell scaffold cartridge comprising a plurality of hollow fibers is depicted. Magnetic clamp 501 is secured to a bioreactor top end 505 through magnetic mechanisms or other approaches. As described herein for various embodiments, a substrate mount 506 can include magnetically responsive materials for securing to one or more adjacent components. In 5b, a substrate mount 506 includes various magnets positioned around the central axis of the mount 506. Substrate mount 506 may be configured to removably engage the substrate portion 507 (in this depiction, a plurality of hollow fibers). Substrate mount 506 can be configured to removably engage the substrate portion 507 through magnetic mechanisms. Hollow tube 508 is depicted in 5b as providing approaches for access to the culture environment surrounding the hollow fibers, and/or for introducing any fluid or other component into the environment.

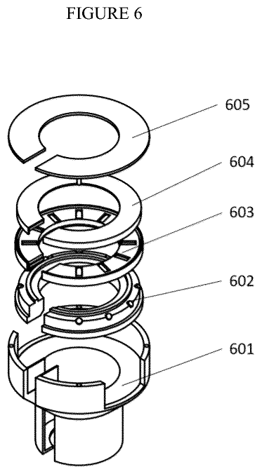

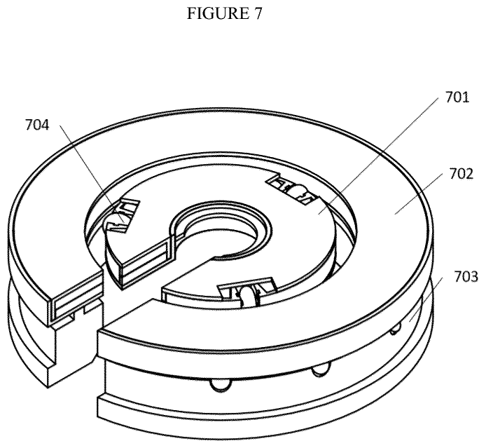

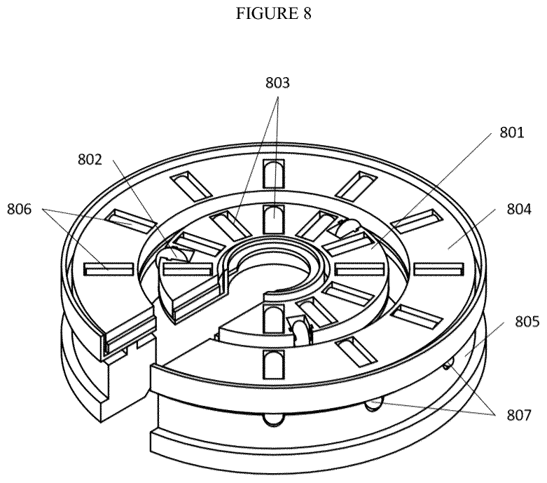

FIG. 6 illustrates an embodiment for an assembly for a bioreactor. The exploded view includes a reactor vessel housing 601 shaped and configured to receive a bioreactor vessel described herein. External ring holders 602 and 603 are depicted and are further illustrated in FIGS. 7 and 8 herein. External ring holder 602 can optionally contain a plurality of magnets arranged circumferentially. The optional plurality of magnets in external holder 602 can be used to shape the magnetic field, which can assist in providing a stabilizing effect on a cell scaffold cartridge (shown in other Figures). Various embodiments can include an external ring holder cap 604 and a housing cap 605. External ring holder cap 604 and housing cap 605 can provide structural support and a protective surrounding or housing for the external ring holders 602 and 603.

FIG. 7 depicts an assembly wherein the substrate mount 701 is positioned within the center of external holders 702 and 703. External ring holder 703 can optionally contain a plurality of magnets arranged circumferentially. The optional plurality of magnets in external holder 703 can be used to shape the magnetic field, which can assist in providing a stabilizing effect to a cell scaffold cartridge (shown in other Figures). Substrate mount 701 is depicted with a plurality of roller bearings 704 that permit it to allow the assembly of inner ring magnets to be maintained at a constant distance from the inner vessel wall and are configured to contact the inner vessel wall for movement along the inner vessel wall.

FIG. 8 is an illustration of the assembly shown in FIG. 7 with a depiction of the internal components of the substrate mount, and external holders. Referring to FIG. 8, substrate mount 801 includes a plurality of roller bearings 802 and a plurality of circumferentially placed magnets 803. Each of upper external holder 804 and lower external holder 805 contain a plurality of circumferentially arranged inner magnets 806, respectively. Inner magnets 806 are, in the embodiment of FIG. 8, hidden and encompassed within the assembly and are received in a recessed magnet holder 807. The inner magnets 806 can be inserted into magnet holder 807 by a snap-fit or by any other mechanism. The circumferentially arranged inner magnets 806 and 807 attract one another to cause the external holders 804 and 805 to be removably secured to one another. In an embodiment, the plurality of magnets in recessed magnetic holder 807 can be arranged to shape the magnetic field, which can assist in stabilizing substrate mount 801.

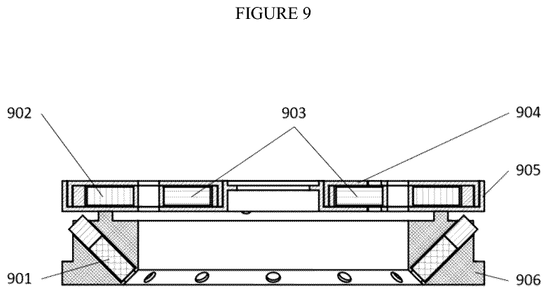

FIG. 9 illustrates a sectional view of the assembly of FIGS. 7 and 8. Lower external holder 906 contains a plurality of circumferentially arranged magnets 901. Upper external ring holder 905 includes a plurality of circumferentially arranged magnets 902. Substrate mount 904 includes a plurality of circumferentially arranged magnets 903. Substrate mount 904 is removably held in place by the magnetic forces between the magnets 903 and the magnets 902 on upper external holder 905. Upper external holder 905 is removably secured to lower external holder 906 by the magnetic attractive forces between magnets 901 and magnets 902. Lower external holder 906 can optionally contain magnets 901 arranged circumferentially to shape the magnetic field and provide a stabilizing effect on the substrate mount.

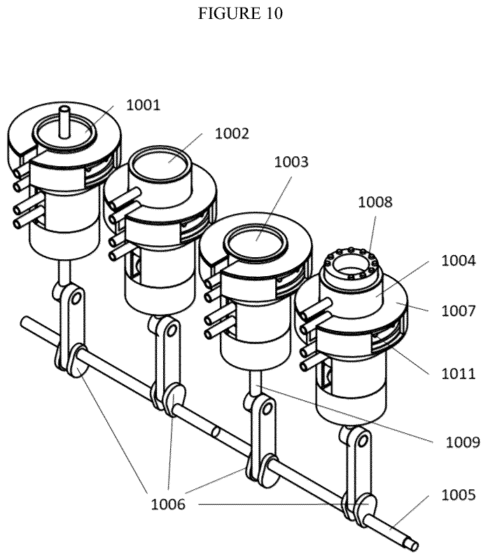

FIG. 10 is an illustration of four different bioreactor assemblies, 1001, 1002, 1003 and 1004, which may be attached to a drive shaft 1005 through a cam mechanism 1006. Each of the four bioreactor assemblies can contain the same or different cell scaffold system and assembly. That is, they can contain a hollow fiber cell scaffold substrate or they can contain a different cell scaffold substrate. Bioreactor housing unit 1007 is depicted with a partial cut-away view illustrating the housing containing the external magnets more detailed elsewhere herein. Magnetic clamp 1008 is depicted on one of the bioreactors 1004 as an example. Magnetic clamp 1008 is of the type depicted elsewhere herein, (e.g., in FIG. 5). In this embodiment, the drive shaft 1005 is rotated axially by a drive mechanism not shown. The drive mechanism can be of any type suitable for rotating drive shaft 1005. As drive shaft 1005 rotates, cam mechanisms 1006 fixed thereto also rotate. Each cam mechanism 1006 can be fixed to a rod mechanism 1009 that is caused to travel in a direction that is substantially along the longitudinal axis of each bioreactor vessel. Rod mechanism 1009 is fixed to a bioreactor vessel 1003. As each rod mechanism 1009 travels, the corresponding and affixed bioreactor vessel 1003 travels in the same direction. External magnets 1011 remain fixed in reactor housing 1007 while the vessel 1003 travels in relation to the external magnets. External magnets 1011 magnetically interact with the substrate mount (e.g., as depicted in FIG. 3 as 303). As a result, the cell scaffold is maintained in a fixed position through magnetic interaction with the external magnets 1011 while the bioreactor vessel 1003 travels relative thereto. Such movement of the components causes the cell scaffold and cells adhered thereto to be at least partially withdrawn from the liquid cell culture medium in the vessel, and thus be exposed to the gaseous components in the head space above the liquid cell culture medium. As further movement of the drive shaft 1005 occurs, the vessel travels bringing the liquid culture medium in further contact with the cell scaffold and adherent cells. This pattern can be repeated through the movement of the drive shaft 1005 at any useful rate. The movement can be stopped, started, and slowed to expose the cell scaffold and cells adherent thereto to any environment, and for any time period.

The various components described herein can be made using any suitable material for the intended purpose, including but not limited to, a polymeric material, metal, alloy, composite, or any combination thereof.

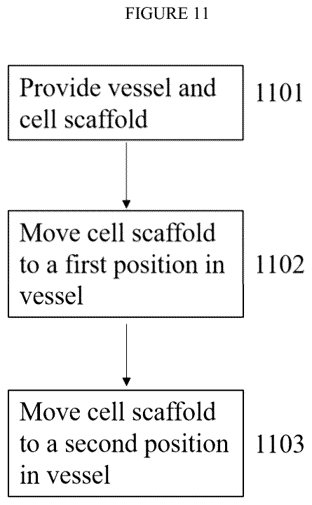

FIG. 11 illustrates an example of a method described herein. The method may comprise the use of an apparatus, such as those depicted in FIGS. 1 and 2, and may comprise any of the components disclosed and described in FIGS. 1-10. Such a method may comprise operation 1101, which can include providing a vessel (e.g., bioreactor) and a cell scaffold comprising at least one cell. In operation 1102, the cell scaffold may be moved to a first position within the vessel to expose the at least one cell to a first fluid or medium (e.g., cell culture media). In operation 1103, the cell scaffold may be moved to a second position within the vessel to expose the at least one cell to a second fluid or medium (e.g., gas).

Within this disclosure, each range of values recited herein includes all combinations and sub-combinations of ranges, as well as specific numerals contained therein. All publications and patent applications cited in this specification are herein incorporated by reference to the extent not inconsistent with the description herein and for all purposes as if each individual publication or patent application were specifically and individually indicated to be incorporated by reference for all purposes.

The foregoing detailed description has set forth various embodiments of the devices and/or processes, and/or examples. Insofar as examples contain one or more functions and/or operations, it will be understood by those within the art that each function and/or operation within such examples can be implemented, individually and/or collectively, by a wide range of hardware, software, firmware, or virtually any combination thereof.

The herein described components (e.g., processes), devices, and objects and the description accompanying them are used as examples for the sake of conceptual clarity and that various configuration modifications using the disclosure provided herein are within the ordinary skill of those in the art. Consequently, as used herein, the specific examples set forth and the accompanying description are intended to be representative of their more general classes. In general, use of any specific example herein is also intended to be representative of its class, and the non-inclusion of such specific components (e.g., processes), devices, and objects herein should not be taken as indicating that limitation is desired.

While the inventive features have been particularly shown and described with reference to preferred embodiments thereof, it will be understood by those in the art that the foregoing and other changes may be made therein without departing from the spirit and the scope of the disclosure. Likewise, the various diagrams may depict an example architectural or other configuration for the disclosure, which is done to aid in understanding the features and functionality that can be included in the disclosure. The disclosure is not restricted to the illustrated example architectures or configurations but can be implemented using a variety of alternative architectures and configurations. Additionally, although the disclosure is described above in terms of various example embodiments and implementations, it should be understood that the various features and functionality described in one or more of the individual embodiments are not limited in their applicability to the particular embodiment with which they are described. They instead can be applied alone or in some combination, to one or more of the other embodiments of the disclosure, whether or not such embodiments are described, and whether or not such features are presented as being a part of a described embodiment. Thus, the breadth and scope of the present disclosure should not be limited by any of the above-described embodiments.

With respect to the use of substantially any plural or singular terms herein, the reader can translate from the plural to the singular or from the singular to the plural as is appropriate to the context or application. The various singular/plural permutations are not expressly set forth herein for sake of clarity.

The herein described subject matter sometimes illustrates different components contained within, or connected with, different other components. It is to be understood that such depicted architectures are merely examples, and that, in fact, many other architectures can be implemented that achieve the same functionality. In a conceptual sense, any arrangement of components to achieve the same functionality is effectively “associated” such that the designated functionality is achieved. Hence, any two components herein combined to achieve a particular functionality can be seen as “associated with” each other such that the designated functionality is achieved, irrespective of architectures or intermediate components. Likewise, any two components so associated can also be viewed as being “operably connected,” or “operably coupled,” to each other to achieve the designated functionality, and any two components capable of being so associated can also be viewed as being “operably couplable,” to each other to achieve the designated functionality. Specific examples of operably couplable include but are not limited to physically mateable or physically interacting components or wirelessly interactable or wirelessly interacting components or logically interacting or logically interactable components.

While particular aspects of the present subject matter described herein have been shown and described, it will be apparent to those in the art that, based upon the teachings herein, changes and modifications may be made without departing from this subject matter described herein and its broader aspects and, therefore, the appended claims are to encompass within their scope all such changes and modifications as are within the true spirit and scope of this subject matter described herein. Furthermore, it is to be understood that the invention is solely defined by the appended claims. In general, terms used herein and especially in the appended claims (e.g., bodies of the appended claims) are generally intended as “open” terms (e.g., the terms “include,” “includes,” or “including” should be interpreted as “including but not limited to,” the term “having” should be interpreted as “having at least”). If a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, one having skill in the art would understand the convention (e.g., “compositions having at least one of A, B, and C” would include but not be limited to, compositions that have A alone, B alone, C alone, A and B together, A and C together, B and C together, and/or A, B, and C together, etc.). In those instances where a convention analogous to “A, B, or C” is used, one having skill in the art would understand the convention (e.g., “a composition having A, B, or C” would include but not be limited to compositions that have A alone, B alone, C alone, A and B together, A and C together, B and C together, and/or A, B, and C together, etc.).

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

While various aspects and embodiments have been disclosed herein, other aspects and embodiments will be apparent to one skilled in the art. The various aspects and embodiments disclosed herein are for purposes of illustration and are not intended to be limiting, with the true scope and spirit being indicated by the following claims.

What is claimed is:

1-68. (canceled)

69. An apparatus comprising:
a) a vessel having an exterior surface and an interior surface defining a sealed cell culture chamber, the vessel having a longitudinal axis, and a plurality of fluid inlet ports and a plurality of fluid outlet ports;
b) a cell scaffold secured to a mount, wherein the mount and the cell scaffold are disposed within the sealed cell culture chamber and configured to move along a longitudinal axis of the vessel, wherein the cell scaffold is not fixed to the sealed cell culture chamber, wherein the mount comprises a magnetically responsive material; and
c) one or more external magnets disposed along the exterior surface of the vessel and configured to magnetically engage the mount upon coupling to the magnetically responsive material.

70. The apparatus of claim 69, wherein the mount and the cell scaffold are configured to be selectively movable between at least a first position and a second position relative to the vessel along the longitudinal axis.

71. The apparatus of claim 69, wherein the vessel is cylindrical in shape and has a top end and a bottom end, wherein the bottom end is removably fixed to a base mechanism, and the top end is removable.

72. The apparatus of claim 71, wherein the base mechanism is configured to move relative to the mount and the cell scaffold.

73. The apparatus of claim 71, wherein the top end of the vessel comprises a magnet and is configured to magnetically engage the mount with a stronger force than the one or more external magnets to secure the mount in the vessel.

74. The apparatus of claim 73, wherein the magnet comprises one or more of an electromagnet, a strong magnet, a permanent magnet, and a neodymium magnet.

75. The apparatus of claim 69, wherein the mount is configured to be selectively movable relative to the vessel between at least a first position and a second position along the longitudinal axis, and wherein movement of the one or more external magnets between the first position and the second position is capable of moving the mount within the sealed cell culture chamber between the first position and the second position.

76. The apparatus of claim 69, wherein the cell scaffold comprises one or more of a plurality of hollow fibers, flat sheet membrane, matrix, cage with macro carriers, and porous membrane bag with macro carriers, or any combination thereof.

77. The apparatus of claim 76, wherein the plurality of hollow fibers, flat sheet membrane, matrix, cage with macro carriers, and porous membrane bag with macro carriers comprise a surface configured to receive cell seeding.

78. The apparatus of claim 76, wherein the plurality of hollow fibers are semipermeable and define an intracapillary space and an extracapillary space, and wherein the intracapillary space is configured to contain a first fluid, and the extracapillary space is configured to contain a second fluid.

79. The apparatus of claim 69, wherein the mount defines a plurality of holes extending therethrough substantially along the longitudinal axis, and wherein the plurality of holes is of a size to permit the transport of a gas or a nutrient therethrough and prevent the transport of a cell therethrough.

80. The apparatus of claim 69, wherein the plurality of fluid inlet ports and the plurality of fluid outlet ports are individually controlled to regulate liquid and gas flow.

81. The apparatus of claim 69, wherein the mount has a top surface and a bottom surface, and the cell scaffold is removably secured to the bottom surface of the mount.

82. The apparatus of claim 69, wherein the vessel further comprises a sampling port configured for aseptically extracting cell culture media.

83. A cell scaffold cartridge comprising:
a mount configured to be disposed within a cell culture chamber of a vessel and move along a longitudinal axis of the vessel, wherein the mount comprises a magnetically responsive material; and
a cell scaffold secured to the mount and configured to move along the longitudinal axis of the vessel, wherein the cell scaffold is not fixed to the cell culture chamber.

84. The cell scaffold cartridge of claim 83, wherein the mount has a top surface and a bottom surface, wherein the cell scaffold is removably secured to the bottom surface of the mount.

85. The cell scaffold cartridge of claim 83, wherein the cell scaffold is one or more of a plurality of hollow fibers, flat sheet membrane, matrix, cage with macro carriers, porous membrane bag with macro carriers, or any combination thereof.

86. The cell scaffold cartridge of claim 85, wherein the plurality of hollow fibers, flat sheet membrane, matrix, cage with macro carriers, and porous membrane bag with macro carriers comprise a surface configured to receive cell seeding.

87. The cell scaffold cartridge of claim 83, wherein the plurality of hollow fibers are semipermeable and define an intracapillary space and an extracapillary space, and wherein the intracapillary space is configured to contain a first fluid, and the extracapillary space is configured to contain a second fluid.

88. The cell scaffold cartridge of claim 83, wherein the mount defines a plurality of holes extending therethrough substantially along the longitudinal axis, and wherein the plurality of holes is of a size to permit the transport of a gas or a nutrient therethrough and prevent the transport of a cell therethrough.
